(19) United States
(12) Patent Application Publication
(10) Pub. No.: US 2006/0156431 A1
(43) Pub. Date: Jul. 13, 2006

(54) L-gulose dependent vitamin C synthesis

(75) Inventor: Beata Wolucka, Brussels (BE)

Correspondence Address: TRASK BRITT, P.O. BOX 2550, SALT LAKE CITY, UT, 84110, US

(73) Assignees: Vlaams Interuniversitair Instituut Voor Biotechnologie VZW, Zwijnaarde (BE); Universiteit Gent, Gent (BE)

(21) Appl. No.: 11/335,247

(22) Filed: Jan. 18, 2006

Related U.S. Application Data
(63) Continuation of application No. PCT/EP04/51415, filed on Jul. 8, 2004.

(30) Foreign Application Priority Data
Jul. 18, 2003 (EP) 03102220.5

(51) Int. Cl. A01H 1/00; C12P 19/02; C12P 17/06; C12N 15/82

(52) U.S. Cl. 800/278; 435/125; 435/105

(57) ABSTRACT

The present invention relates to L-gulose dependent vitamin C synthesis. More specifically, the present invention relates to a synthesis pathway of vitamin C, comprising the formation of GDP-L-gulose by GDP-mannose 3″,5″-epimerase, and subsequent transformation into L-gulose and L-ascorbic acid.

### CROSS-REFERENCE TO RELATED APPLICATIONS

This application is a continuation of PCT International Patent Application No. PCT/EP2004/051415, filed on Jul. 8, 2004, designating the United States of America, and published, in English, as PCT International Publication No. WO 2005/014844 A1 on Feb. 17, 2005, which application claims priority to European Patent Application No. 03102220.5 filed Jul. 18, 2003, the entire contents of each of which are incorporated by this reference.

### TECHNICAL FIELD

The present invention relates to L-gulose dependent vitamin C synthesis. More specifically, the present invention relates to a synthesis pathway of vitamin C, comprising the formation of GDP-L-gulose by GDP-mannose 3″,5″-epimerase, and subsequent transformation into L-gulose and L-ascorbic acid.

### BACKGROUND

Vitamin C (L-ascorbic acid; L-AA) acts as an enzymic cofactor and an antioxidant. In plants, it may represent one of the major soluble carbohydrates and is involved in crucial physiological processes, such as biosynthesis of the cell wall, phytohormones, and secondary metabolites, cell division and growth, stress resistance and photoprotection (1). Large variations in vitamin C content (from 0.003% to 1% of fresh weight; w/w), reported for different plant species, organs and tissues (2), are intimately linked to the vitamin biosynthesis, stability and function. Plants, algae and the majority of animals are able to synthesize vitamin C. Humans, however, lack L-gulono-1,4-lactone oxidase, the last enzyme of the vitamin C pathway in animals, and require L-AA as an essential micronutrient. L-AA biosynthetic genes can be engineered to increase vitamin C content of plants (3-4) in view of improving the nutritional value and stress resistance of crops, but also potentially exploited for the industrial production of vitamin C (5).

The biosynthesis of vitamin C in plants is not completely elucidated and its regulation is largely unknown. Two distinct pathways for vitamin C biosynthesis in plants were proposed (6-7). The salvage pathway involves pectin-derived D-galacturonic acid (D-GalUA)

(6) that is reduced at C1 to L-galactonic acid by the recently identified D-GalUA reductase (4), and the resulting L-galactono-1,4-lactone is oxidized to L-AA by the mitochondrial L-galactono-1,4-lactone dehydrogenase (8-9). Conversion of D-GalUA into L-galactonic acid results in the inversion of carbon numbering. However, labeling studies of Loewus et al. (10) indicated that a non-inversion pathway, in which a hexose is converted into L-AA without reversion of the carbon chain, predominates in plants. The second pathway (7) is a non-inversion, energy-dependent biosynthesis that involves the conversion of GDP-D-mannose to GDP-L-galactose catalyzed by a GDP-D-Man 3″,5″-epimerase (11). L-Galactose, released from the nucleotide through some poorly understood steps, is then oxidized at C1 to L-galactono-1,4-lactone by an L-galactose dehydrogenase (12); the latter compound is converted to vitamin C by the L-galactono-1,4-lactone dehydrogenase.

Recently, we obtained a highly purified GDP-Man 3″,5″-epimerase preparation from Arabidopsis thaliana cell suspensions and identified the corresponding gene (11). Only one copy of the epimerase gene is present in the Arabidopsis genome and the gene is highly conserved among plant species (>88% identity at the protein level and >78% identity at the DNA level). Database search revealed the presence of the epimerase sequence in cDNA libraries obtained from mature tomato fruits and potato tubers but also from salt- and pathogen-stressed ice plant and potato leaves, respectively.

### SUMMARY OF THE INVENTION

Surprisingly, we found that this GDP-Man 3″,5″-epimerase catalyzes a unique double epimerization reaction, preceding the committed step in the biosynthesis of L-AA and glycoconjugates, which results in the irreversible hydrolysis of the highly energetic glycosyl-PP linkage. Therefore, the epimerization step must be tightly controlled. Here, we show that the GDP-Man 3″,5″-epimerase of A. thaliana undergoes a complex regulation that is linked to the cell wall biosynthesis and the redox state of the cell. Even more surprisingly, we demonstrated that Vitamin C is synthesized using a novel intermediate, GDP-L-gulose.

A first aspect of the invention is the use of L-gulose for the synthesis of vitamin C. Preferably, the synthesis comprises the transformation of L-gulose into L-gulono-1,4-lactone. Even more preferably, the transformation is carried out enzymatically, using a dehydrogenase. Preferably, the synthesis comprises also the direct transformation of L-gulono-1,4-lactone into L-ascorbic acid. Even more preferably, the direct transformation is carried out enzymatically, preferably using an L-gulonolactone dehydrogenase. The presence of L-gulonolactone dehydrogenase has been proved in plants. Alternatively, the transformation may be carried out by an L-gulonolactone oxidase. The transformation by L-gulonolactone oxidase is the last step of the vitamin C biosynthesis in animals. “Direct transformation”, as used herein, does not mean that no reaction intermediates are formed; it means, however, that, starting from L-gulono-1,4-lactone, no reaction intermediates are formed belonging to the GDP-L-galactose vitamin C synthesis pathway, such as L-galactono-1,4-lactone.

The synthesis can be a chemical synthesis, using pure chemical reactions, or it can be a biochemical synthesis, using enzymatic transformations. Preferably, the synthesis is a biochemical synthesis. The enzymatic transformation may be carried out in vitro, by isolated enzymes, or by immobilized enzymes, or even by cell systems, or it may be carried out in vivo, either by cells or by whole organisms. Cells include both prokaryotic cells and eukaryotic cells and may be any cell know to the person skilled in the art, and include but are not limited to mammalian cells, yeast cells, fungal cells and plant cells. Preferably, the cells are eukaryotic cells, even more preferably, the cells are plant cells. Organisms may be any organism known to the person skilled in the art, and include, as a non-limiting example, animals and plants. Preferably, the organisms are plants.

All enzymes, needed for the synthesis may be present originally in the cells and/or organism. Alternatively, one or more genes encoding for the enzymes needed for the synthesis are transformed in the cell and/or organism, to allow the synthesis using L-gulose. L-gulose may be added to the culture medium, where it is taken up by the cells and transformed into vitamin C.

Another aspect of the invention is the use of L-gulose according to the invention, whereby L-gulose itself is synthesized in situ. In situ, as used here means that L-gulose is synthesized in the same reaction mixture in which it is used for further transformation. The same reaction mixture, as used here, includes also any cell fraction. Indeed, it is known that several cells, such as the bacteria Thermoplasmum acidophilum (Archeobacteria), Streptomyces verticullus (Actynomycetales) and the algae Volvox and Chlorophyta do produce gulose. The gulose may be further transformed into vitamin C. Preferably, the synthesis of L-gulose comprises the epimerization of GDP-mannose, using GDP-mannose 3″,5″-epimerase. This epimerase reaction may be assisted by a heat shock protein, such as E. coli DnaK or A. thaliana Hsc70.3. Even more preferably, the reaction product of epimerase is GDP-L-gulose. Interaction of the epimerase with the heat shock protein may shift the equilibrium of the epimerase reaction towards the production of GDP-L-gulose.

Preferably, the synthesis comprises the transformation of GDP-L-gulose into L-gulose. Even more preferably, the transformation comprises the use of a protein comprising SEQ ID NO:1, preferably essentially consisting of SEQ ID NO:1, more preferably consisting of SEQ ID NO:1 and/or the use of a protein comprising SEQ ID NO:2, preferably essentially consisting of SEQ ID NO:2, more preferably consisting of SEQ ID NO:2.

This in situ synthesis of L-gulose may be an in vitro synthesis or an in vivo synthesis. Preferably, L-gulose is synthesized in vivo. The in vivo synthesis may be carried out by enzymes that are endogenously present in the cells, of by enzymes that are encoded by genes which have been transformed or transfected to the cell. Indeed, all enzymes to transform the common compound GDP-mannose into L-gulose have been cloned and characterized, and the genes can be transferred to organisms that do not comprise these enzymes endogenously.

Therefore, another aspect of the invention is the use of GDP-Mannose 3″,5″-epimerase to transform GDP-mannose into GDP-L-gulose. Preferably, the GDP-mannose 3″,5″-epimerase is consisting of SEQ ID NO:3. Even more preferably, the GDP-mannose 3″,5″-epimerase is interacting with a heat shock protein, such as E. coli DnaK or A. thaliana Hsc70.3. The interaction of the epimerase with the heat shock protein may shift the equilibrium of the epimerase reaction towards the production of GDP-L-gulose.

Still another aspect of the invention is a method to produce L-gulose starting from GDP-L-gulose, comprising the use of a protein comprising SEQ ID NO:1, preferably essentially consisting of SEQ ID NO:1, even more preferably consisting of SEQ ID NO:1.

Still another aspect of the invention is a method to produce L-gulose starting from GDP-L-gulose, comprising the use of a protein comprising SEQ ID NO:2, preferably essentially consisting of SEQ ID NO:1, even more preferably consisting of SEQ ID NO:2.

### BRIEF DESCRIPTION OF THE FIGURES

FIG. 1. Characterization of GDP-Man 3″,5″-epimerase of A. thaliana. (A) Steady-state parameters of the native (purified from A. thaliana cell suspensions) and recombinant enzyme. The hydroxylapatite fraction of native epimerase (11) was used. The affinity-purified recombinant enzymes were used. (B) SDS-PAGE of the affinity-purified recombinant His-tagged epimerase. Proteins were visualized by Coomassie blue staining. (Left) Molecular mass standards. (Right) 0.3 M imidazole-eluted fraction from Ni-NTA sepharose. Proteins were identified by nano-ESI-MS/MS of in-gel tryptic digests. (C) Competitive inhibition of the native epimerase by GDP. Double reciprocal plots are shown and each line represents a fixed GDP concentration: 0 μM, circles; 2 μM, triangles; and 4 μM, squares. V0 is pmol of GDP-L-Gal/L-Gul produced per minute. GDP-Man concentrations were from 1.1 to 5.5 μM. Inset: Secondary plot of slopes versus GDP concentration was used to determine the Ki value for GDP. (D) Partial inhibition of the native epimerase by GDP-L-Fuc. Incubations contained GDP-[¹⁴C]Man (3.4 μM), increasing amounts of GDP-L-Fuc, and an aliquot of the hydroxylapatite fraction of epimerase. Kinetic measurements were performed in duplicates; standard deviation was equal or less than 5%.

FIG. 2. Formation of GDP-L-gulose by GDP-Man 3″,5″-epimerase of A. thaliana. HPLC analysis of the reaction products obtained with (A) the affinity-purified and (B) a crude (90% ammonium sulfate fraction) GST-tagged recombinant epimerase. (A) The affinity-purified epimerase (3 μg) was incubated with GDP-[¹⁴C]Man. The reaction products at the equilibrium were analyzed either directly as sugar nucleotides (a) or first submitted to mild-acid hydrolysis, followed by the conversion of the released [¹⁴C]sugars to the corresponding PMP derivatives and HPLC analysis at pH 7 (b) and pH 5 (c). The elution of unlabeled sugar standards is indicated. At pH 5, the PMP-Gal derivative elutes at 75 minutes (data not shown). (B) The GST-epimerase-containing crude fraction (90% ammonium sulfate precipitate; 75 μg) was incubated with GDP-[¹⁴C]Man for different intervals of time. A half of each reaction mixture was analyzed directly by HPLC (left). The other half was acid hydrolyzed, the obtained [¹⁴C]hexoses were converted to the corresponding PMP derivatives, and their relative ratios were determined by HPLC (right), as described above.

FIG. 3. Dissection of the GDP-Man 3″,5″-epimerase reaction: possible paths for reversible interconversions between GDP-D-Man, GDP-L-Gul, and GDP-L-Gal. In Scheme 1, the first step is a 5″-epimerization of GDP-D-Man and, thus, the resulting GDP-L-Gul is an obligate intermediate in the formation of GDP-L-Gal from GDP-D-Man. In Scheme 2, GDP-L-Gul and GDP-L-Gal are formed independently. The 5″-epimerization of GDP-D-Man leads to the formation of GDP-L-Gul. The 3″-epimerization of GDP-D-Man would lead to GDP-D-altrose (which was undetectable and, thus, is shown in brackets), followed by the 5″-epimerization of the latter and formation of GDP-L-Gal. A possible interconversion between GDP-L-Gul and GDP-L-Gal through a 3″-epimerization reaction is shown by dotted arrows.

FIG. 4. L-gulose pathway in the de novo biosynthesis of vitamin C in plants. The scheme shows a dual role of GDP-Man 3″,5″-epimerase that converts GDP-D-Man into GDP-L-Gal and GDP-L-Gul. Interactions of the epimerase with a molecular chaperone(s) could increase the enzyme activity and favor the formation of GDP-L-Gul, thus linking the vitamin biosynthesis to stress response. GDP-L-Gul is then channeled exclusively to the vitamin C path: after release from the nucleotide, L-Gul is oxidized to L-AA with L-gulono-1,4-lactone as an intermediate.

### DETAILED DESCRIPTION OF THE INVENTION

### EXAMPLES

Materials and Methods to the Examples

Reagents. D-[U-14C]Man (specific activity 286 mCi/mmol) and guanosine diphospho-D-[U-14C]Man were purchased from Amersham Pharmacia Biotech (Little Chalfont, Buckinghamshire, UK). Ni-NTA superflow resin was obtained from Qiagen (Hilden, Germany).

GST-affinity resin was from Stratagene (Madison, Wis.). All reagents were of analytical grade.

Guanosine diphospho-L-fucose, guanosine diphospho-D-glucose, adenosine diphospho-D-glucose, L-gulose, and L-gulono-1,4-lactone were purchased from Sigma-Aldrich (St. Louis, Mo.).

Plant material. Arabidopsis thaliana (L.) Heynh. ecotype Columbia cell suspensions were grown as described (13). White potato (Solanum tuberosum L. cv. Irish Cobbler) tubers were stored at 13° C. until use.

Plasmids. The GATEWAY™ (Invitrogen, Gaithersburg, Md.) plasmids, pDEST15_Epim and pDEST17_Epim, containing the GDP-Man 3″,5″-epimerase gene of A. thaliana, were prepared as described (11) for the bacterial expression of glutathione S-transferase (GST)- and His-tagged epimerase (N-terminal fusions), respectively.

GDP-Man 3″,5″-epimerase assay and L-AA determination. The GDP-Man 3″,5″-epimerase activity and L-AA were measured by the HPLC method as described (13), with the exception that the concentration of methanol in solvent A was 0.5% and the flow rate was 0.8 ml/min.

In vivo labeling of A. thaliana cell suspensions with D-[U-14C]Man. In vivo labeling of A. thaliana cells was performed as described (13). Cell suspensions were pre-adapted to labeling conditions for 20 hours in the presence or absence of exogenous L-AA or its precursors (2.5 mM), followed by two hours labeling with 1 μCi of D-[U-14C]Man. L-AA was extracted with 5% metaphosphoric acid containing 2 mM DTT and 1 mM EDTA.

Bacterial expression of the recombinant epimerase. Heterologous expression of the recombinant epimerase in Escherichia coli submitted to a “reversed” heat-shock (a shift from 37° C. to 26° C., just before the induction) was performed as described (11). Cells were re-suspended in three volumes of 50 mM Tris-HCl buffer (pH 7.7) containing 0.5 mM DTT, 1 mM PMSF, and 20% glycerol (buffer A). Crude extracts and 90% ammonium sulfate precipitates were prepared as described (11).

Ni-NTA metal-affinity chromatography of the recombinant GDP-Man 3″,5″-epimerase. A crude extract containing the His-tagged epimerase protein was loaded on a 2-ml Ni-NTA superflow column equilibrated with 5 mM imidazole in 25 mM Tris-HCl buffer (pH 7.7) containing 1 mM PMSF (buffer B). The column was washed with ten volumes of the equilibration buffer, followed by five volumes of 20 mM imidazole in buffer B. The elution was carried out with three volumes of 300 mM imidazole in buffer B.

GST-affinity chromatography of the recombinant GDP-Man 3″,5″-epimerase. A crude extract containing the GST-tagged epimerase was applied to a 2-ml GST-affinity column equilibrated with buffer A. The column was washed with 15 volumes of buffer A and the recombinant epimerase was eluted with three volumes of 10 mM glutathione (reduced form) in buffer A.

Extraction and assay of L-gulono-1,4-lactone dehydrogenase activity. The L-gulono-1,4-lactone dehydrogenase activity was extracted from white potato tubers essentially as described (14), except that gel filtration was performed on NAP-25 columns (Amersham Pharmacia Biotech) and the obtained high-molecular weight fraction was separated by centrifugation (20,000×g for 20 minutes) into the “cytosolic” (supernatant) and the “mitochondrial” (pellet) fractions. The dehydrogenase activity was measured spectrophotometrically at 550 nm by following the L-gulono-1,4-lactone-dependent reduction of cytochrome c (14).

PAGE. Proteins were separated by SDS/PAGE, using 12.5% minigels and the buffer system described by Laemmli (15). Gels were stained with Coomassie brilliant blue R-250.

Peptide sequencing and protein identification. Tryptic peptides prepared from in-gel digested protein bands were analyzed by nano-electrospray tandem mass spectrometry, and the obtained sequence information was submitted to database searching, as described (11).

Protein determination. Protein concentration was determined by the method of Bradford (16), using BSA as standard.

Sugar analysis. GDP-[14C]hexoses of the epimerase reaction mixtures were hydrolyzed in 50 mM HCl at 100° C. for 20 minutes. For HPLC analysis, the acid-released [14C]hexoses together with cold sugar standards were converted to the corresponding 1-phenyl-3-methyl-5-pyrazolone (PMP) derivatives (17). The HPLC system with on-line UV and radioactivity detection (13) was used. The solvent was 18% acetonitrile in 0.1 M phosphate buffer pH 7.0 or pH 5.0 for separation of PMP derivatives of Man and Gul, at a flow rate of 0.8 ml/min. To identify altrose, free sugars were separated by TLC on silica gel 60 aluminum sheets (pre-impregnated with 0.3 M NaH2PO4) in acetone/n-butanol/water (8:1:1, v/v/v) and detected as described (13).

### Example 1

### Characterization of the GDP-Man 3″,5″-Epimerase

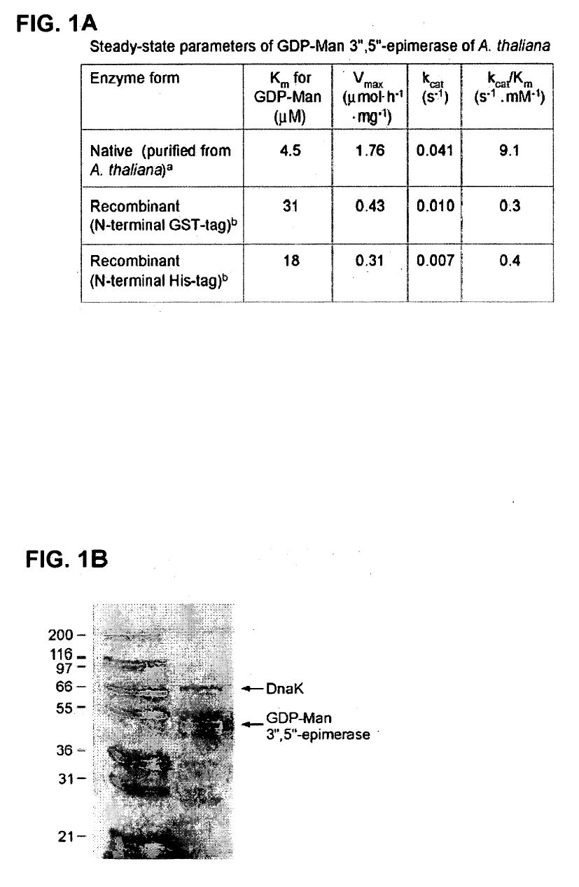

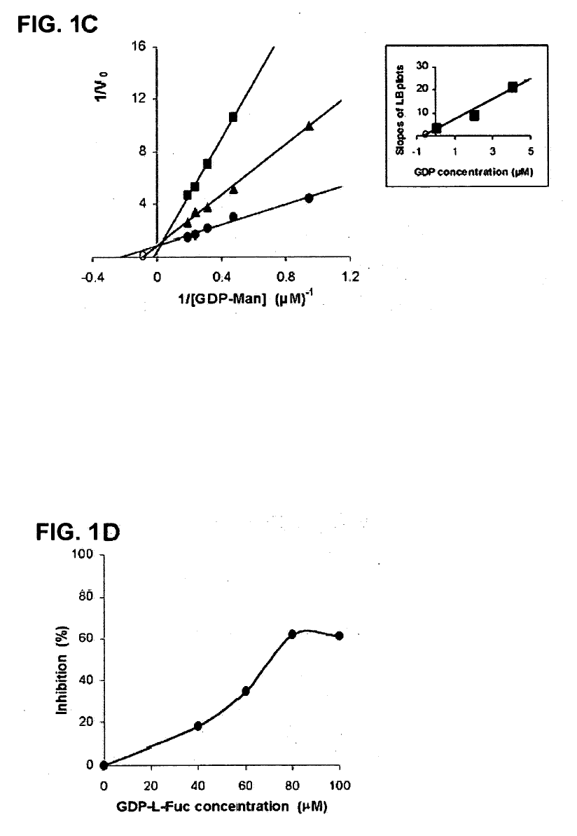

To gain insight into the regulation of the de novo biosynthesis of vitamin C, we have characterized the native and recombinant epimerase of A. thaliana. The epimerase belongs to the short-chain dehydrogenase/reductase family (18). The native enzyme is a homodimer of a 43-kDa subunit (11) and possesses two potential NAD-binding sites and two potential substrate-binding sites per dimer (19). The epimerase has a low Km for the GDP-Man substrate (4.4 μM) (FIG. 1A) comparing to the Chlorella epimerase (96 μM) (20) and to the related bacterial enzymes, GDP-Man 4″,6″-dehydratase (19) and GDP-L-Fuc synthetase (21-22) (280 μM and 38.6 μM, respectively). In contrast to the GDP-Man 4″,6″-dehydratase (23) and GDP-L-Fuc synthetase (24) of E. coli, the epimerase is strongly inhibited by GDP and GDP-Glc in a competitive manner, with respective Ki of 0.7 μM (FIG. 1C) and 5 μM (data not shown). The enzyme recognizes the purine moiety of GDP-derivatives because an adenosine derivative, ADP-D-Glc, had no effect on the enzyme activity. Surprisingly, only a partial inhibition was observed with GDP-L-Fuc (150=70 μM; FIG. 1D), even at 1 mM concentration. The sigmoidal inhibition curve with GDP-L-Fuc (FIG. 1D) is reminiscent of a feedback regulation observed in the biosynthesis of NDP-6-deoxyhexoses in bacteria (25-27). Like CDP-Glc 4″,6″-dehydratase of Yersinia pseudotuberculosis (28), the purified epimerase was stimulated by exogenously added oxidized forms of nicotinamide-adenine dinucleotides, NAD+ and NADP+ (145 and 110% of control, respectively), and inhibited by their reduced forms, NADH and NADPH (78% and 88% of control, respectively). Physiological concentrations (1 mM) of L-AA, a reducing agent and the end product of the pathway, inhibited the epimerase activity by 15%. In agreement, a feedback inhibition of the L-AA biosynthesis was clearly observed in vivo, because feeding A. thaliana cells with exogenous L-AA resulted in an increased level of the intracellular L-AA and a decreased incorporation of the [14C]Man label into L-AA (Table 1). The partial inhibition of the epimerase by L-AA might be explained in terms of “reductive inhibition” (29), i.e., as an L-AA-dependent reduction of the enzyme-NAD+complex in the absence of the nucleotide sugar. However, the same degree of inhibition (14%) was observed in the presence of 1 mM L-galactono-1,4-lactone, whereas D-isoascorbic acid and L-galactose had no effect. These facts suggest the existence of a stereospecific mechanism of the enzyme inhibition by sugar lactones.

We cloned the epimerase gene of A. thaliana (11) and affinity-purified the recombinant His-and GST-tagged proteins from the E. coli host submitted to a “reversed” heat shock. Of the recombinant epimerase protein (3 mg per liter of culture), 90% were found in the soluble fraction, but only 30% of the total activity were retained on the affinity columns, probably because of interactions with other proteins. Analysis of the affinity-purified enzyme by SDS-PAGE (FIG. 1B), followed by MS identification of the protein bands, revealed the presence of the major GDP-Man 3″,5″-epimerase band and of a weak 70-kDa band, corresponding to DnaK, a Hsp70 chaperone of E. coli (the tryptic peptides identified are TTPSIIAYTQDGETLVGQPAK (SEQ ID NO:4), IINEPTAAALAYGLDK (SEQ ID NO:5), and SLGQFNLDGINPAPR (SEQ ID NO:6)). Thus, the recombinant epimerase could interact with an Hsp70 molecular chaperone. During purification of the native GDP-Man 3″,5″-epimerase from A. thaliana cell suspensions (11), a 70-kDa chaperone (DnaK ortholog) co-purified with the epimerase throughout the whole procedure and was identified in the NaCl-eluate from Blue-Sepharose as a cytosolic Hsc70.3 heat-shock cognate protein of Arabidopsis (At3gO9440; the identified tryptic peptides are NQVAMNPINTVFDAK (SEQ ID NO:7), NAVVTVPAYFNDSQR (SEQ ID NO:8), DAGVIAGLNVMR (SEQ ID NO:9), VQQLLVDFFNGK (SEQ ID NO:10), and FELSGIPPAPR (SEQ ID NO:11)). The majority of the Hsc70 protein was separated from the epimerase by gel filtration. This step resulted in a tenfold decrease of the epimerase activity, possibly because of not only the partial loss of the NAD cofactor (11), but also the disruption of interactions with the Hsc70 chaperone.

The steady-state parameters of the recombinant GST- and His-tagged epimerase were comparable to those of the native enzyme, although the recombinant enzymes were catalytically less efficient (FIG. 1A). The Vmax of the native enzyme was low, suggesting a rate-limiting enzyme. The overall catalytic efficiency of GDP-Man 3″,5″-epimerase expressed as kcat/Km (9.1 s-1 mM-1) was fairly good and comparable to that reported for the GDP-L-Fuc synthetase of E. coli (21).

### Example 2

### Formation of GDP-L-gulose by the GDP-Man 3″,5″-epimerase of A. thaliana

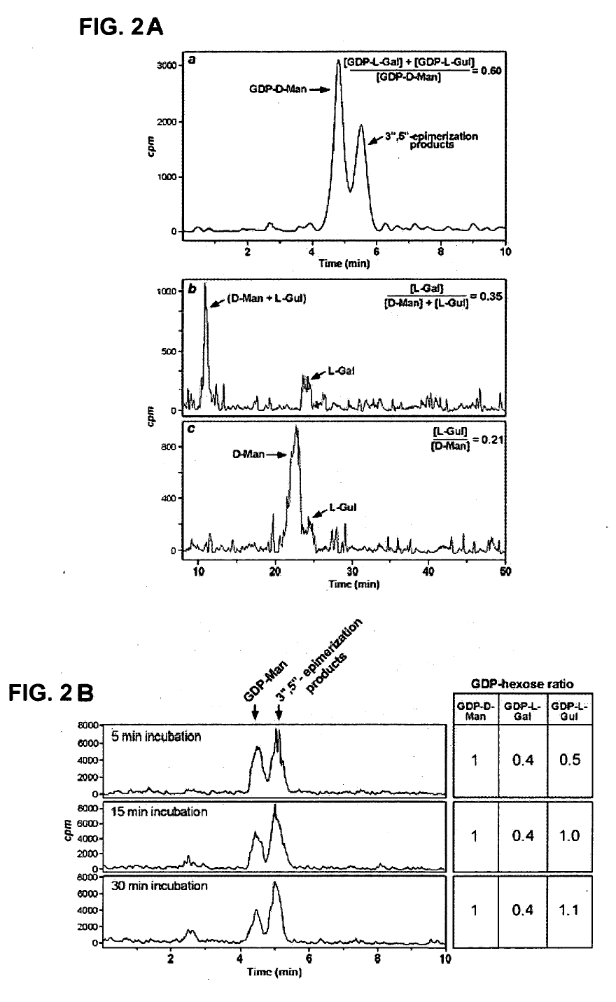

The most intriguing observation was the variation of the apparent equilibrium constant K′eq (from 0.1 to 1.5) measured for different preparations of the recombinant epimerase. Similar variations of K′eq, but in a narrower range (from 0.1 to 0.4), were observed with preparations of the native epimerase from A. thaliana. Also, an unexplained anomaly with the measured K′eq values for the reverse reaction were reported for the epimerase of Chlorella sp. (20). FIG. 2A (panel a) shows the HPLC profile of the reaction products at the equilibrium obtained with the affinity-purified epimerase. The measured ratio (K′eq) of the epimerization product(s) to the GDP-Man substrate was 0.6. If GDP-L-Gal were the only epimerization product, then a similar ratio (0.6) should be obtained for the mild-acid-released [14C]labeled L-Gal versus D-Man. The measured L-Gal to D-Man ratio was only 0.35 (FIG. 2A, panel b), fact that indicated that the Man peak contained an unknown component. This component was separated from D-Man and co-migrated with L-gulose standard (FIG. 2A, panel c). Therefore, we conclude that the epimerase reaction mixture contained at the equilibrium GDP-D-Man, GDP-L-Gal, and GDP-L-Gul in a respective ratio of 1:0.4:0.2. A similar analysis of the epimerization products obtained with an epimerase-containing 55-70% ammonium sulfate fraction from A. thaliana cell suspensions revealed the presence of GDP-D-Man, GDP-L-Gal, and GDP-L-Gul in a ratio of 1:0.18:0.09.

FIG. 2B shows the HPLC profiles of the epimerase reaction products obtained with a crude recombinant enzyme (90% ammonium sulfate fraction) and the relative ratios of the GDP-hexoses formed. In this case, the reaction was shifted towards the GDP-L-Gul formation and the relative ratios of GDP-D-Man, GDP-L-Gal and GDP-L-Gul at the equilibrium were 1:0.4:1.1 (FIG. 2B).

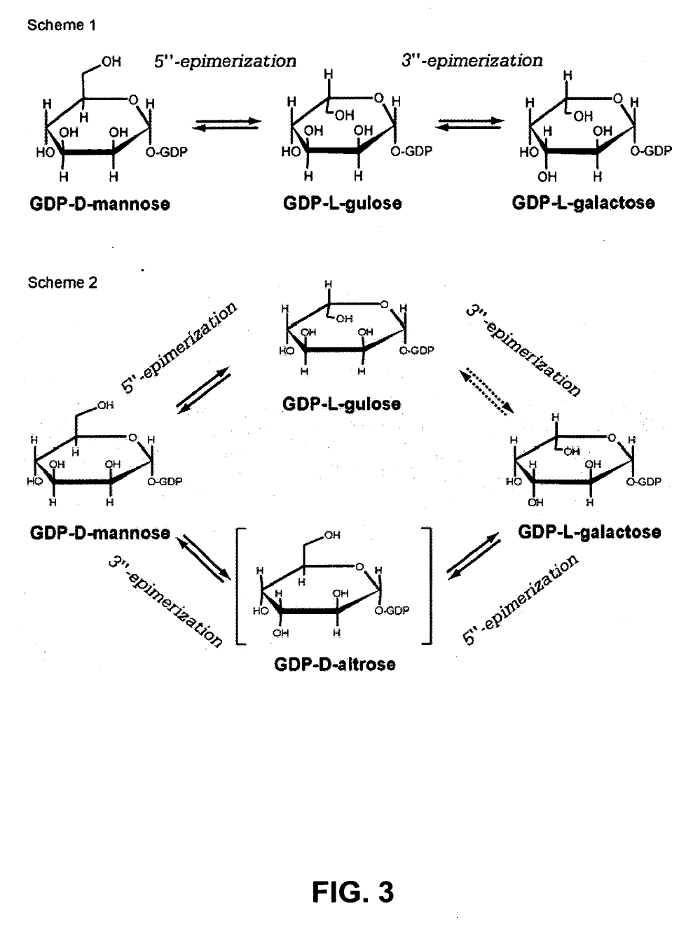

Our results demonstrate that the GDP-Man 3″,5″-epimerase reaction can be dissected into at least two distinct epimerization reactions leading to the formation of two discrete products: GDP-L-Gal and GDP-L-Gul (FIG. 3). The fate of the epimerization seems to depend on the molecular form of the enzyme, probably as a result of its interactions with molecular chaperones.

### Example 3

### Synthesis of L-AA from L-Gulose and L-gulono-1,4-Lactone by Plant Cells

In contrast to L-Gal, which is a minor constituent of plants, L-Gul, as far as we know, has never been reported in plants. Therefore, GDP-L-Gul might be dedicated to the biosynthesis of L-AA. To test this hypothesis, we supplied A. thaliana cells with cold L-Gul or L-gulono-1,4-lactone and measured the level of cold L-AA in the cells. As reported before for cress seedlings, preincubation with L-gulono-1,4-lactone (30) but also with L-Gul resulted in an increased level of vitamin C, and L-gulono-1,4-lactone was as efficient precursor of L-AA as was L-galactono-1,4-lactone (Table 1). Moreover, in the presence of cold precursors, a decreased incorporation of the [14C]Man label into vitamin C was observed (Table 1), as expected for biosynthetic intermediates. L-AA inhibited its own biosynthesis (Table 1) by a feedback mechanism apparently at the level of GDP-Man 3″,5″-epimerase.

Therefore, we demonstrated that L-Gul and L-gulono-1,4-lactone are converted into L-AA by A. thaliana cell suspensions. The L-Gal dehydrogenase can use L-Gul as substrate (12). However, the mitochondrial L-galactono-1,4-lactone dehydrogenase is highly specific and does not oxidize L-gulono-1,4-lactone (8-9). Therefore, A. thaliana cells must possess another enzyme able to convert L-gulono-1,4-lactone to L-AA. L-Gulono-1,4-lactone dehydrogenase activity was reported in the cytosolic fraction of Euglena sp. (31) and in the mitochondrial fraction obtained from potato tubers (14). In agreement with Ôba et al. (14), we could detect the L-gulono-1,4-lactone dehydrogenase activity (0.66 mU/g of tissue) in the mitochondrial fraction from potato tubers. This activity represented about 30% of that of L-galactono-1,4-lactone dehydrogenase measured with L-galactono-1,4-lactone as substrate (2.16 mU/g of tissue). Surprisingly, the majority (75%) of L-gulono-1,4-lactone dehydrogenase activity (4.51 mU/g of tissue) was found in the “cytosolic fraction” of potato tubers, which points to the existence of differently localized isozymes.

### Example 4

### Regulation of the GDP-Man 3″ 5″-Epimerase

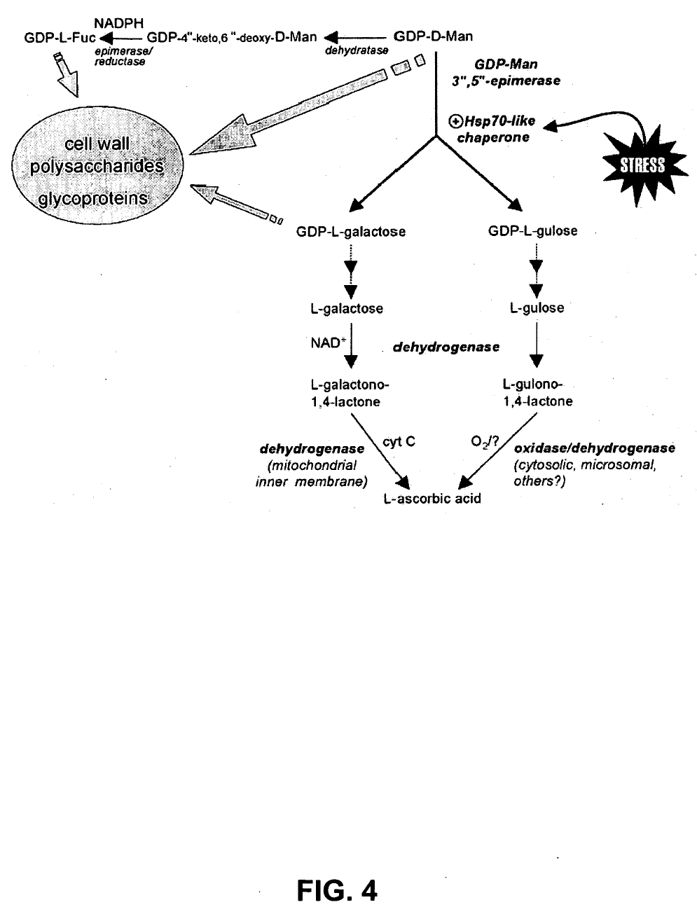

The biochemical characterization of the GDP-Man 3″,5″-epimerase of A. thaliana has brought new insights into the de novo biosynthesis of L-AA and its regulation. The unexpected partial inhibition of the epimerase by GDP-L-Fuc (FIG. 1D) could be of paramount importance because, even at high concentration of GDP-L-Fuc in the cell, the epimerase would still supply GDP-L-Gal/GDP-L-Gul substrates necessary for the de novo synthesis of L-AA. The complex type of inhibition by GDP-L-Fuc could also play a role in the regulation of the cell-wall biosynthesis in plants. In the presence of high concentrations of GDP-L-Fuc, the cellular level of the GDP-L-Gal precursor will be low, thus resulting in a lesser incorporation of L-Gal into glycoconjugates. Indeed, in the L-Fuc-deficient mur1 mutant of Arabidopsis, which lacks the GDP-Man 4″,6″-dehydratase activity (32) catalyzing the first step in GDP-L-Fuc pathway (FIG. 4), L-Gal replaces L-Fuc in xyloglucans (33) and N-glycans (34).

We observed that the epimerase is stimulated by the oxidized forms of nicotinamide adenine dinucleotides (NAD+/NADP+), but inhibited by their reduced forms (NADH/NADPH) and by L-AA. Because the NAD-binding motif (GAGGFI) present within the epimerase sequence is a modified version of the common Rossmann consensus (GxGxxG) found in other members of the short-chain dehydrogenase/reductase family (35), this feature could be responsible for a lower affinity of the enzyme for the dinucleotides and the corresponding stimulation/inhibition effects. Moreover, our in vivo experiments demonstrated a feedback inhibition in the vitamin C pathway (Table 1). We propose, therefore, that the GDP-Man 3″,5″-epimerase catalyzing the first specific step in the biosynthesis of vitamin C could sense the redox state of the cell and play an important role in the regulation of vitamin C and cell-wall/glycoproteins biosynthesis.

### Example 5

### Role of Heat Shock Protein in the Epimerase Reaction

Both recombinant and native GDP-Man 3″,5″-epimerase of A. thaliana co-purify with Hsp70 heat-shock proteins (E. coli DnaK and A. thaliana Hsc70.3, respectively) and different molecular forms of the enzyme could be detected on the basis of its chromatographic behavior and enzymatic properties (K′eq). These facts indicate that the Hsc70 protein and possibly other molecular chaperones could be implicated in the regulation of the epimerase activity and favor the formation of GDP-L-Gul. The Hsc70 heat-shock protein of Arabidopsis is constitutively expressed and stress-inducible (37). The highly-conserved, ubiquitous Hsp70 chaperones play a key role in protection and adaptation to stress by participating in folding and unfolding of misfolded and native-state proteins; targeted delivery of proteins to specific cellular domains and organelles (38); disassembly of regulatory complexes (39); and regulation of protein/enzyme activity (40-44). Interactions of the epimerase with Hsp70 proteins may represent a molecular basis of the increased vitamin C level of Arabidopsis leaves in response to heat shock (45) and be implicated in the salt- and heat-tolerance of transgenic tobacco overexpressing the bacterial DnaK chaperone (46-47).

TABLE 1
Effect of exogenous L-ascorbic acid (L-AA) and precursors on
its de novo biosynthesis and cellular content in A. thaliana
cell suspensions*

| | Cold L-AA | [¹⁴C]-labeled L-AA |
|---|---|---|
| | nmol/g fw | 10⁻³ × cpm/mg fw |
| Compound | (% of control) | (% of control) |
| Control (no addition) | 212 (100) | 1342 (100) |
| L-Gulose | 403 (190) | 872 (65) |
| L-Galactose | 668 (315) | 423 (32) |
| L-Galactono-1,4-lactone | 360 (170) | 790 (59) |
| L-Gulono-1,4-lactone | 345 (165) | 722 (54) |
| L-AA | 1764 (832) | 448 (33) |

*4-day-old A. thaliana cell suspensions were pre-incubated for 20 hours with 2.5 mM L-AA or its precursors, and then labeled for two hours with D-[U-¹⁴C]Man. [¹⁴C]-labeled and cold total L-AA in acid extracts was measured by HPLC. fw, fresh weight.

### REFERENCES

1. Smimoff N. and G. L. Wheeler (2000) Crit. Rev. Plant Sci. 19, 267-290.
2. Davies M. B., J. Austin and D. A. Partridge (1991) Vitamin C: Its Chemistry and Biochemistry (Royal Society of Chemistry, Cambridge), p. 81.
3. Jain A. K. and C. G. Nessler (2000) Mol. Breeding 6, 73-78.
4. Agius F., R. González-Lamothe, J. L. Caballero, J. Muñoz-Blanco, M. A. Botella and V. Valpuesta (2003) Nature Biotech. 21, 177-181.
5. Hancock R. D. and R. Viola (2002) Trends Biotechnol. 20, 299-305.
6. Isherwood F. A., Y. T. Chen and L. W. Mapson (1953) Nature 171, 348-349.
7. Wheeler G. L., M. A. Jones and N. Smirnoff (1998) Nature 393, 365-369.
8. Ostergaard J., G. Persiau, M. W. Davey, G. Bauw and M. Van Montagu (1997) J. Biol. Chem. 272, 30009-30016.
9. Imai T., S. Karita, G.-I. Shiratori, M. Hattori, T. Nunome, K. Õba and M. Hirai (1998) Plant Cell Physiol. 39, 1350-1358.
10. Loewus F. A. and S. Kelly (1961) Nature 191, 1059-1061.
11. Wolucka B. A., G. Persiau, J. Van Doorsselaere, M. W. Davey, H. Demol, J. Vandekerckhove, M. Van Montagu, M. Zabeau and W. Boerjan (2001) Proc. Natl. Acad. Sci. USA 98, 14843-14848.
12. Gatzek S., G. L. Wheeler and N. Smimoff (2002) Plant J. 30, 541-553.
13. Wolucka B. A., M. Davey and W. Boerjan (2001) Anal. Biochem. 294, 161-168.
14. Ôba K., M. Fukui, Y. Imai, S. Iriyama and K. Nogami (1994) Plant Cell Physiol. 35, 473-478.
15. Laemmli U. K. (1970) Nature 227, 680-685.
16. Bradford M. M. (1976) Anal. Biochem. 72, 248-254.
17. Honda S., E. Akao, S. Suzuki, M. Okuda, K. Kakehi and J. Nakamura (1989) Anal. Biochem. 180, 351-357.
18. Jörnvall H., B. Persson, M. Krook, S. Atrian, R. González-Duarte, J. Jeffery and D. Ghosh (1995) Biochemistry 34, 6003-6013.
19. Somoza J. R., S. Menon, H. Schmidt, D. Joseph-McCarthy, A. Dessen, M. L. Stahl, W. S. Somers and F. X. Sullivan (2000) Structure 8, 123-135.
20. Hebda P. A., E. J. Behrman and G. A. Barber (1979) Arch. Biochem. Biophys. 194, 496-502.
21. Rosano C., A. Bisso, G. Izzo, M. Tonetti, L. Sturla, A. De Flora and M. Bolognesi (2000) J. Mol. Biol. 303, 77-91.
22. Somers W. S., M. L. Stahl and F. X. Sullivan (1998) Structure 6, 1601-1612.
23. Sturla L., A. Bisso, D. Zanardi, U. Benatti, A. De Flora and M. Tonetti (1997) FEBS Letters 412, 126-130.
24. Menon S., M. Stahl, R. Kumar, G.-Y. Xu and F. Sullivan (1999) J. Biol. Chem. 274, 26743-26750.
25. Melo A. and L. Glaser (1965) J. Biol. Chem. 240, 398-405.
26. Kornfeld R. H. and V. Ginsburg (1966) Biochim. Biophys. Acta 117, 79-87.
27. Blankenfeldt W., M. Asuncion, J. S. Lam and J. H. Naismith (2000) EMBO J. 19, 6652-6663.
28. He X., J. S. Thorson and H.-W. Liu (1996) Biochemistry 35, 4721-4731.
29. Gabriel O. (1978) Trends Biochem. Sci. 3, 193-195.
30. Isherwood F. A., Y. T. Chen and L. W. Mapson (1954) Biochem. J. 56, 1-15.
31. Shigeoka S., Y. Nakano and S. Kitaoka (1979) Agric. Biol. Chem. 43, 2187-2188.
32. Bonin C. P., I. Potter, G. F. Vanzin and W.-D. Reiter (1997) Proc. Natl. Acad. Sci. USA 94, 2085-2090.
33. Zablackis E., W. S. York, M. Pauly, S. Hantus, W.-D. Reiter, C. C. S. Chapple, P. Albersheim and A. Darvill (1996) Science 272, 1808-1810.
34. Rayon C., M. Cabanes-Macheteau, C. Loutelier-Bourhis, I. Salliot-Maire, J. Lemoine, W.-D. Reiter, P. Lerouge and L. Faye (1999) Plant Physiol. 119, 725-733.
35. Scrutton N. S., A. Berry and R. N. Perham (1990) Nature 343, 38-43.
36. Allard S. T. M., M.-F. Giraud and J. H. Naismith (2001) Cell. Mol. Life Sci. 58, 1650-1665.
37. Wu S.-H., C. Wang, J. Chen and B.-L. Lin (1994) Plant Mol. Biol. 25, 577-583.
38. Hartl F. U. and M. Hayer-Hartl (2002) Science 295, 1852-1858.
39. Freeman B. C. and K. R. Yamamoto (2002) Science 296, 2232-2235.
40. Pratt W. B., P. Krishna and L. J. Olsen (2001) Trends Plant Sci. 6, 54-58.
41. Morishima Y., P. J. M. Murphy, D.-P. Li, E. R. Sanchez and W. B. Pratt (2000) J. Biol. Chem. 275, 18054-18060.
42. Haus U., P. Trommler, P. R. Fisher, H. Hartmann, F. Lottspeich, A. A. Noegel and M. Schleicher (1993) EMBO J. 12, 3763-3771.
43. Lutz W., K. Kohno and R. Kumar (2001) Biochem. Biophys. Res. Commun. 282, 1211-1219.
44. Yan W., C. L. Frank, M. J. Korth, B. L. Sopher, I. Novoa, D. Ron and M. G. Katze (2002) Proc. Natl. Acad. Sci. USA 99, 15920-15925.
45. Panchuk I. I., R. A. Volkov and F. Schöffl (2002) Plant Physiol. 129, 838-853.
46. Sugino M., T. Hibino, Y. Tanaka, N. Nii, T. Takabe and T. Takabe (1999) Plant Sci. 137, 81-88.
47. Ono K., T. Hibino, T. Kohinata, S. Suzuki, Y. Tanaka, T. Nakamura, T. Takabe and T. Takabe (2001) Plant Sci. 160, 455-461.

1. A method of synthesizing vitamin C, said method comprising utilizing L-gulose for synthesis of vitamin C.

2. The method according to claim 1, wherein the synthesis comprises transformation of L-gulose to L-gulono-1,4-lactone.

3. The method according to claim 2, wherein said L-gulono-1,4-lactone is directly transformed into L-ascorbic acid by a dehydrogenase.

4. The method according to claim 1, wherein said synthesis is a biosynthesis.

5. The method according to claim 4, wherein said biosynthesis is carried out in a plant.

6. The method according to claim 2, wherein said synthesis is a biosynthesis.

7. The method according to claim 6, wherein said biosynthesis is carried out in a plant.

8. The method according to claim 3, wherein said synthesis is a biosynthesis.

9. The method according to claim 8, wherein said biosynthesis is carried out in a plant.

10. The method according to claim 1, wherein L-gulose itself is synthesized in situ.

11. The method according to claim 6, wherein the synthesis of L-gulose comprises epimerization of GDP-mannose, utilizing GDP-mannose 3″,5″-epimerase.

12. The method according to claim 6, wherein the synthesis of L-gulose comprises the transformation of GDP-L-gulose into L-gulose.

13. The method according to claim 7, wherein the synthesis of L-gulose comprises the transformation of GDP-L-gulose into L-gulose.

14. A method of transforming GDP-mannose into GDP-L-gulose, said method comprising using GDP-Mannose 3″,5″-epimerase to transform the GDP-mannose into GDP-L-gulose.

15. The method according to claim 14, wherein said GDP-Mannose 3″,5″-epimerase is interacting with a protein of the heat shock protein 70 family

16. A process for producing L-gulose starting from GDP-L-gulose, said process comprising utilizing a protein comprising SEQ ID NO: 1 and/or SEQ ID NO:2 in said process.

17. The process of claim 16, wherein the protein comprises SEQ ID NO:1.

18. The process of claim 16, wherein the protein comprises SEQ ID NO:2.


SEQUENCE LISTING (13 sequences)

SEQ ID NO: 1 - protein, 529 aa, Arabidopsis thaliana
MARNFLLVLLWFIVQVSSSHENGRGDQALSQIDIYAINLAQHHSAFIHVSPLVLGSQYMYAKSSPDYMKTGNAVLKFMLINQRADFSFALFTGGLSNPTLVSVSNHVSFINPKAPVYPRLALGKKWDEMTVTWTSGYNIGEAVPFVEWSRKGTRSRRSPAGTLTFTRNSMYTYRMGHELMNGSIVWSKNFTFKSSPYPGQDSLQRVIIFGDMGKGERDGSNEYNDYQPGSLNTTDQLIKDLKNIDIVFHIGDITYANGYISQWDQFTAQVEPIASTVPYMVASGNHERDWPDSGSFYGGKDSGGECGVPAETMFDFPAENKAKFWYSADYGMFRFCVADTEHDWREGSEQYQFIERCLASVDRRAQPWLIFIAHRVLGYSTNDWYGQEGSFEEPMGRESLQKLWQKYKVDIAFYGHVHNYERTCPIYQNQCMDNEKSHYSGAFKGTIHVVVGGAGSHLSSFSSLKPKWSIFRDYDYGFVKLTAFDHSSLLFEYKKSSNGAVHDSFTIFREYRDVLACVRDSCEPTTLAS

SEQ ID NO: 2 - protein, 280 aa, Arabidopsis thaliana
MEFENRYGLATSQKYDCLLFDLDDTLYPLSSGIARECGNNIKDYMTEKLGIPKDKIVELSDLLYKNYGTTMAGLRAIGYEFDYDEYHSFVHGRLPYDNIKPDLVLRSLLLSLPLRKVIFTNADRVHAAKALKKLGLEDCFEGIICFETLNLMHTNAASNNSEIFDIVGHFNRSEPVGSLPKTPVVCKPSESAIEKALEIANIDPNRTLFFEDSVRNVQAGKRVGLYTVLVGKSTKVKGADYALENIHNMKEAIPELWESDRKSSDVGYSGKVAVETSVRA

SEQ ID NO: 3 - protein, 377 aa, Arabidopsis thaliana
MGTTNGTDYGAYTYKELEREQYWPSENLKISITGAGGFIASHIARRLKHEGHYVIASDWKKNEHMTEDMFCDEFHLVDLRVMENCLKVTEGVDHVFNLAADMGGMGFIQSNHSVIMYNNTMISFNMIEAARINGIKRFFYASSACIYPEFKQLETTNVSLKESDAWPAEPQDAYGLEKLATEELCKHYNKDFGIECRIGRFHNIYGPFGTWKGGREKAPAAFCRKAQTSTDRFEMWGDGLQTRSFTFIDECVEGVLRLTKSDFREPVNIGSDEMVSMNEMAEMVLSFEEKKLPIHHIPGPEGVRGRNSDNNLIKEKLGWAPNMRLKEGLRITYFWIKEQIEKEKAKGSDVSLYGSSKVVGTQAPVQLGSLRAADGKE

SEQ ID NO: 4 - protein, 21 aa, Arabidopsis thaliana
TTPSIIAYTQDGETLVGQPAK

SEQ ID NO: 5 - protein, 16 aa, Arabidopsis thaliana
IINEPTAAALAYGLDK

SEQ ID NO: 6 - protein, 15 aa, Arabidopsis thaliana
SLGQFNLDGINPAPR

SEQ ID NO: 7 - protein, 15 aa, Arabidopsis thaliana
NQVAMNPINTVFDAK

SEQ ID NO: 8 - protein, 15 aa, Arabidopsis thaliana
NAVVTVPAYFNDSQR

SEQ ID NO: 9 - protein, 12 aa, Arabidopsis thaliana
DAGVIAGLNVMR

SEQ ID NO: 10 - protein, 12 aa, Arabidopsis thaliana
VQQLLVDFFNGK

SEQ ID NO: 11 - protein, 11 aa, Arabidopsis thaliana
FELSGIPPAPR

SEQ ID NO: 12 - protein, 6 aa, Artificial Sequence
  NAD-binding motif
GAGGFI

SEQ ID NO: 13 - protein, 6 aa, Artificial Sequence
  Rossmann consensus
GXGXXG